(19) United States
(12) Patent Application Publication
(10) Pub. No.: US 2026/0033774 A1
(43) Pub. Date: Feb. 5, 2026

(54) Sleep parameters estimation using millimeter-wave radar

(71) Applicant: Neteera Technologies Ltd., Jerusalem (IL)

(72) Inventors: Segev COHEN, Herzliya (IL); Alex KAZAKOV, Jerusalem (IL); Yair DEITCHER, Efrat (IL); Dana SHAVIT, Mevasseret Zion (IL)

(21) Appl. No.: 19/352,511

(22) Filed: Oct. 8, 2025

Related U.S. Application Data
(63) Continuation-in-part of application No. PCT/IL2024/050370, filed on Apr. 16, 2024.
(60) Provisional application No. 63/705,048, filed on Oct. 9, 2024.

(51) Int. Cl. A61B 5/00; A61B 5/0205; A61B 5/0507; A61B 5/08; A61B 5/11; A61B 5/113

(52) U.S. Cl. CPC A61B 5/4812; A61B 5/0205; A61B 5/0507; A61B 5/08; A61B 5/1102; A61B 5/113; A61B 5/4809; A61B 5/4818; A61B 5/725; A61B 5/7257; A61B 5/7282

(57) ABSTRACT

A system and method for monitoring sleep parameters, the method including during a model training phase: receiving millimeter-wave reflection radar signals from reference subjects, extracting in-phase and quadrature components, deriving displacement signals reflecting body micromovements from cardiac and pulmonary activity, segmenting displacement signals into reference segments, forming a training dataset with segments labeled with measured sleep data, and applying machine learning to generate a sleep parameters estimation model; and during a subject monitoring phase: receiving millimeter-wave reflection radar signals from a monitored subject, extracting signal components, deriving displacement signals reflecting body micromovements, segmenting into monitored segments, applying the estimation model to estimate sleep parameters, determining overall sleep duration, and determining a sleep parameters index based on estimated parameters and duration.

### CROSS-REFERENCE TO RELATED APPLICATIONS

This application is a Continuation-in-Part application of PCT International Application No. PCT/IL2024/050370, filed on Apr. 16, 2024, and this application claims the benefit of U.S. Provisional Patent Application 63/705,048, filed on Oct. 9, 2024, both of which are hereby incorporated by reference in their entirety.

### TECHNICAL FIELD

The present disclosure generally relates to the fields of high frequency radar sensors, temporal signal processing, machine learning, and sleep monitoring.

### BACKGROUND

Sleep apnea is a common sleep disorder characterized by repeated interruptions in breathing during sleep. These breathing disruptions, which include complete cessations of airflow (apneas) and partial reductions in airflow (hypopneas), can occur multiple times throughout the night and significantly impact sleep quality and overall health. The condition affects millions of people worldwide, with prevalence rates increasing with age and certain risk factors such as obesity.

Sleep stages represent distinct phases of sleep characterized by specific patterns of brain activity, eye movement, and muscle tone. Sleep is generally divided into rapid eye movement (REM) sleep and non-rapid eye movement (NREM) sleep, with NREM sleep further subdivided into three stages. Understanding sleep architecture and the time spent in different sleep stages provides valuable insights into sleep quality and can help identify various sleep disorders.

Traditional diagnosis and monitoring of sleep disorders typically rely on polysomnography (PSG), which involves overnight monitoring at specialized sleep laboratories. This approach requires multiple sensors and electrodes to be attached to the patient's body to measure various physiological parameters including brain activity, eye movements, muscle activity, cardiac function, and respiratory effort. While PSG remains the gold standard for sleep disorder diagnosis, it presents several limitations including patient discomfort, the artificial sleep environment of a laboratory setting, high costs, and limited availability.

The cumbersome nature of traditional sleep monitoring systems has driven interest in developing more convenient and accessible alternatives. Contactless monitoring technologies offer the potential to assess sleep parameters without requiring physical sensors attached to the body, thereby enabling more natural sleep conditions and facilitating long-term monitoring in home environments.

Radar-based sensing technologies have emerged as a promising approach for contactless physiological monitoring. These systems can detect minute body movements and physiological signals by analyzing reflected electromagnetic waves. High frequency radar systems may offer particular advantages for biological sensing applications due to their ability to penetrate clothing and bedding materials while providing high sensitivity to small-scale movements associated with cardiac and respiratory activity.

Machine learning techniques have shown considerable promise in analyzing complex physiological signals and extracting meaningful health-related information. These approaches can identify patterns and relationships in data that may not be readily apparent through traditional signal processing methods, potentially enabling more accurate and reliable assessment of sleep parameters from contactless sensor data.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for monitoring sleep parameters is provided. The method includes during a model training phase, for each of a plurality of reference subjects: receiving a millimeter-wave reflection radar signal reflected from a respective one of the reference subjects, sampling the reflection radar signal and extracting a signal portion at a range of the respective one of the reference subjects, the signal portion consisting of an in-phase component and a quadrature component, deriving a displacement signal reflecting body micromovements associated with cardiac and pulmonary activity from the extracted signal portion, segmenting the displacement signal into reference subject segments having a selected segment duration, forming a training dataset including training samples with each training sample including a respective reference subject segment labeled with measured sleep data during the segment duration, and applying machine learning processes to the training dataset to generate a sleep parameters estimation model. The method includes during a subject monitoring phase: receiving a millimeter-wave reflection radar signal reflected from a monitored subject, sampling the reflection radar signal and extracting a signal portion at a range of the monitored subject, the signal portion consisting of an in-phase component and a quadrature component, deriving a displacement signal reflecting body micromovements associated with cardiac and pulmonary activity from the extracted signal portion, segmenting the displacement signal into monitored subject segments having the selected segment duration, applying the sleep parameters estimation model to the monitored subject segments to estimate sleep parameters over a monitoring period, determining an overall sleep duration of the monitored subject during the monitoring period, and determining a sleep parameters index of the monitored subject based on the estimated sleep parameters and the determined sleep duration.

According to other aspects of the present disclosure, the method may include one or more of the following features. The sleep parameters may include at least one of: sleep apnea events; and sleep stage classifications. The sleep parameters index may include at least one of: an apnea-hypopnea index calculated as a total number of detected apnea and hypopnea events divided by the overall sleep duration; and a sleep stage distribution index. The method may further include processing the displacement signals prior to segmenting, using at least one of: bandpass filtering; normalization; and down-sampling. The method may include applying bandpass filtering to the displacement signals to preserve frequencies between 0.05 Hz and 3.33 Hz. The reflection radar signal may be sampled at a sampling rate of 500 Hz and the displacement signal may be down-sampled to 10 Hz prior to the segmenting. The displacement signals may be derived from the extracted signal portions through non-linear filtering and mapping operations applied to the in-phase and quadrature components. Determining the overall sleep duration may include detecting a sleep status of the monitored subject to distinguish between sleep and wake states during the monitoring period. The millimeter-wave reflection radar signal may include a frequency-modulated continuous wave (FMCW) radar signal. Extracting a signal portion may include applying a fast Fourier transform (FFT) to the FMCW radar signal. The millimeter-wave reflection radar signal may be at a frequency above 100 GHz. The method may further include establishing classification profiles by categorizing reference subjects based on demographic and physiological characteristics, and applying different model parameters for different classification profiles during sleep parameter estimation.

According to another aspect of the present disclosure, a system for monitoring sleep parameters is provided. The system includes a radar device configured to receive a millimeter-wave reflection radar signal reflected from each of a plurality of reference subjects during a model training phase, and to receive a millimeter-wave reflection radar signal reflected from a monitored subject during a subject monitoring phase. The system includes a processor configured to, during the model training phase, for each of the plurality of reference subjects: sample the reflection radar signal reflected from the respective one of the reference subjects and extract a signal portion at a range of the respective one of the reference subjects, the signal portion consisting of an in-phase component and a quadrature component; derive a displacement signal reflecting body micromovements associated with cardiac and pulmonary activity from the extracted signal portion; segment the displacement signal into reference subject segments having a selected segment duration; form a training dataset including training samples, each training sample including a respective reference subject segment labeled with measured sleep data during the segment duration; and apply machine learning processes to the training dataset to generate a sleep parameters estimation model. The processor is configured during the subject monitoring phase to: sample the reflection radar signal reflected from the monitored subject and extract a signal portion at a range of the monitored subject, the signal portion consisting of an in-phase component and a quadrature component; derive a displacement signal reflecting body micromovements associated with cardiac and pulmonary activity from the extracted signal portion; segment the displacement signal into monitored subject segments having the selected segment duration; apply the sleep parameters estimation model to the monitored subject segments to estimate sleep parameters of the monitored subject over a monitoring period; determine an overall sleep duration of the monitored subject during the monitoring period; and determine a sleep parameters index of the monitored subject based on the estimated sleep parameters and the determined sleep duration.

According to other aspects of the present disclosure, the system may include one or more of the following features. The sleep parameters may include at least one of: sleep apnea events; and sleep stage classifications. The sleep parameters index may include at least one of: an apnea-hypopnea index calculated as a total number of detected apnea and hypopnea events divided by the overall sleep duration; and a sleep stage distribution index. The processor may be further configured to process the displacement signal prior to segmenting, using at least one of: bandpass filtering; normalization; and down-sampling. The processor may be configured to determine the overall sleep duration by detecting a sleep status of the monitored subject to distinguish between sleep and wake states during the monitoring period. The radar device may be configured to receive the millimeter-wave reflection radar signal as a frequency-modulated continuous wave (FMCW) radar signal. The processor may be configured to establish classification profiles by categorizing reference subjects based on demographic and physiological characteristics, and apply different model parameters for different classification profiles during sleep parameter estimation.

The foregoing general description of the illustrative embodiments and the following detailed description thereof are merely exemplary aspects of the teachings of this disclosure and are not restrictive.

### BRIEF DESCRIPTION OF THE DRAWINGS

The present disclosure will be more fully understood from the following detailed description of the embodiments thereof, taken together with the drawings in which:

FIG. 1 illustrates a schematic diagram of a sleep parameters monitoring system, according to aspects of the present disclosure;

FIG. 2 illustrates a radar device obtaining radar reflection signals from a patient on a bed, according to aspects of the present disclosure;

FIG. 3 illustrates a block diagram of a sleep parameters monitoring method, according to aspects of the present disclosure;

FIG. 4 illustrates a flow diagram of a data acquisition and model training phase of the sleep parameters monitoring method, according to aspects of the present disclosure; and

FIG. 5 illustrates a flow diagram of a subject monitoring phase of the sleep parameters monitoring method, according to aspects of the present disclosure.

### DETAILED DESCRIPTION OF EMBODIMENTS

The following description sets forth exemplary aspects of the present disclosure. It should be recognized, however, that such description is not intended as a limitation on the scope of the present disclosure. Rather, the description also encompasses combinations and modifications to those exemplary aspects described herein.

### Terms/Definitions

Unless otherwise defined, all terms (including technical and scientific terms) used herein have the same meaning as commonly understood by one of ordinary skill in the art to which the disclosed subject matter belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on”, “attached” to, “operatively coupled” to, “operatively linked” to, “operatively engaged” with, “connected” to, “coupled” with, “contacting”, “added” to, another element, it can be directly on, attached to, connected to, operatively coupled to, operatively engaged with, coupled with, added to, and/or contacting the other element or intervening elements can also be present. In contrast, when an element is referred to as being “directly contacting” another element or “directly added” to another element, there are no intervening elements present.

Whenever the term “about” or “approximately” is used, it is meant to refer to a measurable value such as an amount, a temporal duration, and the like, and is meant to encompass variations (e.g., ±20%, ±10%, ±5%, ±1%, ±0.1%) from the specified value, as such variations are appropriate to perform the disclosed methods.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Throughout, this disclosure mentions “disclosed embodiments”, “disclosed systems” and “disclosed methods”, which refer to examples of inventive ideas, concepts, and/or manifestations described herein. The fact that some disclosed embodiments are described as exhibiting a feature or characteristic does not mean that other disclosed embodiments necessarily share that feature or characteristic.

This disclosure employs open-ended permissive language, indicating for example, that some embodiments “may” employ, involve, or include specific features. The use of the term “may” and other open-ended terminology is intended to indicate that although not every embodiment may employ the specific disclosed feature, at least one embodiment employs the specific disclosed feature.

The term “repeatedly” as used herein should be broadly construed to include any one or more of: “continuously”, “periodic repetition” and “nonperiodic repetition”, where periodic repetition is characterized by constant length intervals between repetitions and non-periodic repetition is characterized by variable length intervals between repetitions.

The terms “patient” and “subject” are used interchangeably herein to refer to an individual upon which at least one aspect of a method or system of the present disclosure is applied, such as a person undergoing monitoring. A patient may be any living entity, such as a person, human or animal, characterized with physiological functioning.

The terms “user” and “operator” are used interchangeably herein to refer to any individual person or group of persons using or operating at least one aspect of a method or system of the present disclosure.

The terms “sleep apnea event” and “apnea event”, are used interchangeably herein to refer to an instance of irregular breathing during sleep, including pauses in breathing (typically referred to as “apneas”), or periods of shallow or reduced breathing (typically referred to as “hypopneas”), where an apnea is defined as a complete absence in the airflow of a sleeping subject for a minimum duration (e.g., at least 10 seconds), and a hypopnea is defined as a reduction in airflow (e.g., by at least 30%) of a sleeping subject for a minimum duration (e.g., at least 10 seconds). A sleep apnea event may include obstructive apneas/hypopneas (i.e., caused by an obstruction or partial blockage in the subject airway), central apneas/hypopneas (i.e., caused by a reduced or failed attempt at breathing that may result from neurological malfunctioning), or a combination of both.

The term “Apnea-Hypopnea Index (AHI)” as used herein refers to a metric representing the average number of apnea and hypopnea events per hour of sleep, which may be used as an indication of the severity of sleep apnea of a subject.

The term “sleep stage” as used herein refers to a discrete time period of sleep which a subject may experience during a sleeping session, such as by progressing cyclically through multiple sleep stages, where a sleep stage may include a rapid eye movement (REM) sleep stage, or a non-rapid eye movement (NREM) sleep stage such as: NREM stage 1 (N1), NREM stage 2 (N2), and NREM stage 3 (N3), or an “awake” state.

### General Overview

The present disclosure relates to systems and methods for contactless and unobtrusive monitoring of sleep parameters using millimeter-wave radar technology combined with machine learning analysis. In some aspects, the disclosed technology may enable accurate estimation of sleep apnea events and/or sleep stages through analysis of physiological micro-movements detected by radar signals reflected from a subject during sleep. The system may utilize radar signals, such as frequency-modulated continuous wave radar operating in Terahertz frequency bands, to capture subtle body displacements associated with cardiac and pulmonary activity. Machine learning models may be trained using radar-derived displacement signals paired with polysomnography reference data to establish predictive relationships between radar signal patterns and sleep parameters. During monitoring phases, the trained models may analyze real-time radar data to provide sleep parameter assessments including apnea-hypopnea index calculations and sleep stage classifications, enabling convenient home-based sleep monitoring without requiring physical sensors attached to the subject body. The present disclosure may provide for accurate and contactless estimation of sleep parameters without requiring a time-consuming calibration process for different environments, for different sleep surfaces, or for different subjects. In some examples, the monitored sleep parameters include sleep apnea information. In some examples, the monitored sleep parameters include sleep stages information.

### System Overview

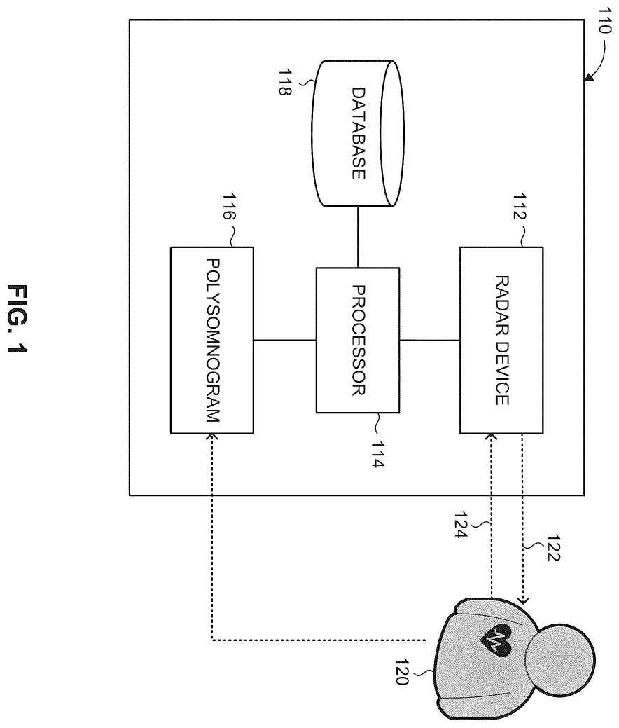

Reference is made to FIG. 1, which illustrates a schematic diagram of a sleep parameters monitoring system, generally referenced 110, according to aspects of the present disclosure. System 110 for monitoring sleep parameters may include multiple interconnected components configured to operate in both training and monitoring phases. System 110 includes a radar device 112, a processor 114, a polysomnogram (PSG) device 116, and a database 118. Processor 114 is communicatively coupled with radar device 112, with PSG device 116, and with database 118, to facilitate data exchange and coordinated operation across the system components. System 110 may be configured to monitor sleep parameters of a subject 120 through contactless radar-based sensing combined with machine learning analysis.

Radar device 112 is configured to transmit a transmitted radar signal 122 toward subject 120 and to receive a reflected radar signal 124 that contains information relating to micro-displacements associated with cardiac and pulmonary activity. Radar device 112 includes at least one radar transmitter and at least one radar receiver. In some cases, radar device 112 may include a multiple-input multiple-output (MIMO) radar configuration with phased array radar capabilities. A MIMO configuration may enable radar device 112 to utilize multiple transmitting and receiving elements, such as individual radar antennas or arrays of radar antennas arranged in a phased array configuration. The phased array radar capabilities may allow for enhanced signal processing and improved detection of physiological micro-movements through coordinated transmission and reception patterns across multiple antenna elements. The transmitted radar signals 122 may be at a sufficiently high frequency to ensure that the signal is reflected and not absorbed by the body tissue, for example in the millimeter wave (MMW) frequency band (corresponding to EHF radio frequencies). According to an embodiment of the present disclosure, the transmitted and reflected radar signals 122, 124 is in the Terahertz (THz) frequency band, where the term “Terahertz (THz)” as used herein encompasses Terahertz and sub-Terahertz radiation corresponding to sub-millimeter and millimeter wave radiation, such as electromagnetic waves within the frequency band between about 0.03 to 3 THz, corresponding to radiation wavelengths between about 10 mm to 0.1 mm. In one example, radar device 112 operates at a frequency above 100 GHz, such as approximately 120 GHz (or 0.12 THz). Radar device 112 may be as described for example in PCT International Publications WO2018/167777A1 and WO2020/012455A1 to Neteera Technologies Ltd, the disclosures of which are incorporated herein by reference.

Processor 114 may receive information and instructions from other components of system 110 and may perform data processing operations. During a data acquisition phase, processor 114 may process the reflected radar signal 124 obtained by radar device 112 to generate machine learning models. During a subject monitoring phase, processor 114 may apply the trained models to determine sleep parameters of the subject 120. In some cases, processor 114 may be distributed across multiple processing units or may be part of a server or remote computer system accessible over communications networks.

PSG device 116 may record physiological information of the subject 120 during sleep monitoring sessions. PSG device 116 may obtain polysomnogram recordings that include readings relating to one or more physiological functions of the subject, such as: cardiac activity through electrocardiography (ECG), brain activity through electroencephalography (EEG), eye movements through electrooculography (EOG), and/or muscle activity through electromyography (EMG). Accordingly, PSG device 116 may be embodied by one or more devices configured to detect physiological functions, such as: an instrument configured to detect cardiac activity (e.g., an ECG device), an instrument configured to detect brain activity (e.g., an EEG device), an instrument configured to detect eye movements or ocular activity (e.g., an EOG device), and/or an instrument configured to detect muscular activity (e.g., an EMG device).

Database 118 may store radar signal data, associated physiological information, and other relevant data to be retrieved and processed by processor 114. In some cases, database 118 may be represented by local servers, or by remote and distributed servers, such as in cloud storage platforms.

Information may be conveyed between the components of system 110 over data communication channels or networks using various transmission protocols including wired, wireless, radio, WiFi, and Bluetooth connections. System 110 may store, manage, and process data using cloud computing infrastructure, allowing components to communicate with one another and be remotely monitored or controlled over the Internet, such as through Internet of Things (IoT) network configurations. The components of system 110 may be based in hardware, software, or combinations thereof. The functionality associated with each component may be distributed among multiple devices or components that may reside at a single location or multiple locations. For example, the functionality associated with processor 114 may be distributed between a single processing unit or multiple processing units (e.g., a dedicated machine learning processor for the data modeling phase). Processor 114 may be part of a server or a remote computer system accessible over a communications medium or network, such as a cloud computing platform. Processor 114 may also be integrated with other components of system 110, such as incorporated with radar device 112.

System 110 may include additional components not shown in FIG. 1 to enable implementation of aspects of the present disclosure, such as to enhance operational capabilities and user interaction. For example, a user interface may be provided for allowing users to control various parameters or settings associated with the components of system 110. A display device may be included for visually displaying information relating to the operation of system 110, such as real-time monitoring data, analysis results, or system status indicators. In some cases, a camera or imaging device may be incorporated for capturing images of the operation of system 110, which may provide supplementary visual information to complement radar-based sensing data. These additional components may be integrated with the core system components or may be provided as separate modules that interface with system 110 through established communication pathways.

### Radar Device and System Deployment

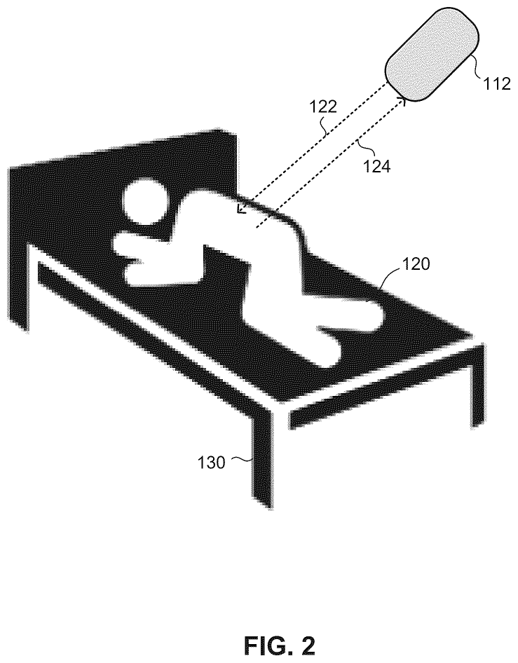

FIG. 2 illustrates a radar device obtaining radar reflection signals from a patient on a bed, according to aspects of the present disclosure. Radar device 112 transmits a coherent transmitted radar signal 122 to a monitoring area, such as a frequency-modulated continuous wave (FMCW) radar signal in the THz band, and receives a reflection radar signal 124 respective of the transmitted radar signal 122, such as a reflection signal reflected from a body part of patient 120. Reflection radar signal 124 may contain information relating to displacements associated with cardiac activity and pulmonary activity. In one example, the monitoring area includes a supporting surface, such as a bed 130, for supporting patient 120. Patient 120 may be in a lying or recumbent position on bed 130, with the body aligned substantially horizontally, such as in a prone posture, a supine posture, or a side posture, while patient 120 is in a sleeping or resting state. Bed 130 may generally encompass various types of supporting surfaces, including but not limited to: a chair; a couch; a sofa; a cot; a crib; a divan; a mattress; a box-spring; and the like.

System 110 may be deployed in practical monitoring environments where subject 120 may be positioned on a bed 130 during sleep monitoring sessions. Radar device 112 may be positioned in various configurations relative to the bed 130 to optimize signal transmission and reception for different monitoring scenarios. For example, radar device 112 may be positioned above bed 130, and directed toward the chest area of subject 120 when subject 120 may be in a supine position. Alternatively, radar device 112 may be positioned under bed 130 and directed toward the back of subject 120 when subject 120 may be in a supine position. Radar device 112 may also be positioned on the side of bed 130 to accommodate different room configurations or monitoring preferences. Radar device 112 may be mounted to a structure in the vicinity of bed 130, such as a wall, a ceiling, or a post. In one example, a single radar device 112 is deployed which has a field of view (FOV) encompassing an entirety of bed 130. Alternatively, system 110 may include a plurality of radar devices 112 having a respective FOV, where the FOVs of the plurality of radar devices 112 encompass an entirety of bed 130. Radar device 112 may be positioned at a predetermined distance from bed 130, such as within several meters. In one example, radar device 112 is positioned up to approximately 150 centimeters (cm) from subject 120.

As shown in FIG. 2, radar device 112 may transmit the transmitted radar signal 122 from various directional approaches relative to subject 120 to accommodate different room layouts and monitoring configurations. For example, transmitted radar signal 122 may be directed from anterior directions, such as toward the chest or abdomen of subject 120, or from posterior directions, such as toward the back, neck or shoulder regions. In some cases, radar device 112 may transmit signals from non-orthogonal angles relative to subject 120, allowing for flexible positioning that may not require precise perpendicular alignment with the subject body. The reflected radar signal 124 may be received by radar device 112 regardless of the transmission angle, providing operational flexibility in real-world deployment scenarios where optimal positioning may be constrained by room furniture, architectural features, or other environmental factors.

The transmitted radar signal 122 and reflected radar signal 124 may exhibit enhanced penetration capabilities that enable monitoring through various material barriers commonly encountered in sleep environments. The radar signals may penetrate through clothing worn by subject 120, including pajamas, nightgowns, or other sleepwear materials that might otherwise obstruct optical or infrared sensing systems. In some cases, the transmitted radar signal 122 may also penetrate through fabric materials of lying surfaces such as bed sheets, blankets, comforters, or mattress materials. Radar device 112 may maintain signal integrity when subject 120 may be covered by bedding materials, allowing for continuous monitoring without requiring direct exposure of the subject body to the sensing system.

System 110 may operate effectively in low light or poor visibility conditions without requiring direct line-of-sight to the subject 120. The radar-based sensing approach may function independently of ambient lighting conditions, enabling continuous monitoring during nighttime hours when traditional optical sensing systems might experience degraded performance. System 110 may maintain monitoring capabilities even when subject 120 may be partially or completely obscured by bedding materials, pillows, or other objects that might block visual observation. Radar device 112 may detect physiological micro-movements and breathing patterns through these obstructions, providing reliable sleep parameter monitoring without environmental lighting constraints or visual accessibility requirements.

System 110 may be configured to simultaneously monitor multiple subjects in a given location using source separation techniques that distinguish between different physiological signal sources. When multiple subjects may be present in the same room or on the same bed 130, processor 114 may apply signal processing algorithms to separate and identify individual physiological signatures from a combined reflected radar signal 124. In some cases, the source separation techniques may analyze signal characteristics such as breathing rates, heart rates, movement patterns, or spatial positioning to differentiate between subjects and provide individual sleep parameter assessments. Radar device 112 may capture reflected signals from multiple subjects simultaneously, and processor 114 may process these combined signals to generate separate monitoring datasets for each subject, enabling comprehensive sleep analysis in shared sleeping environments or clinical settings with multiple patients.

### Sleep Parameters Monitoring Method Overview

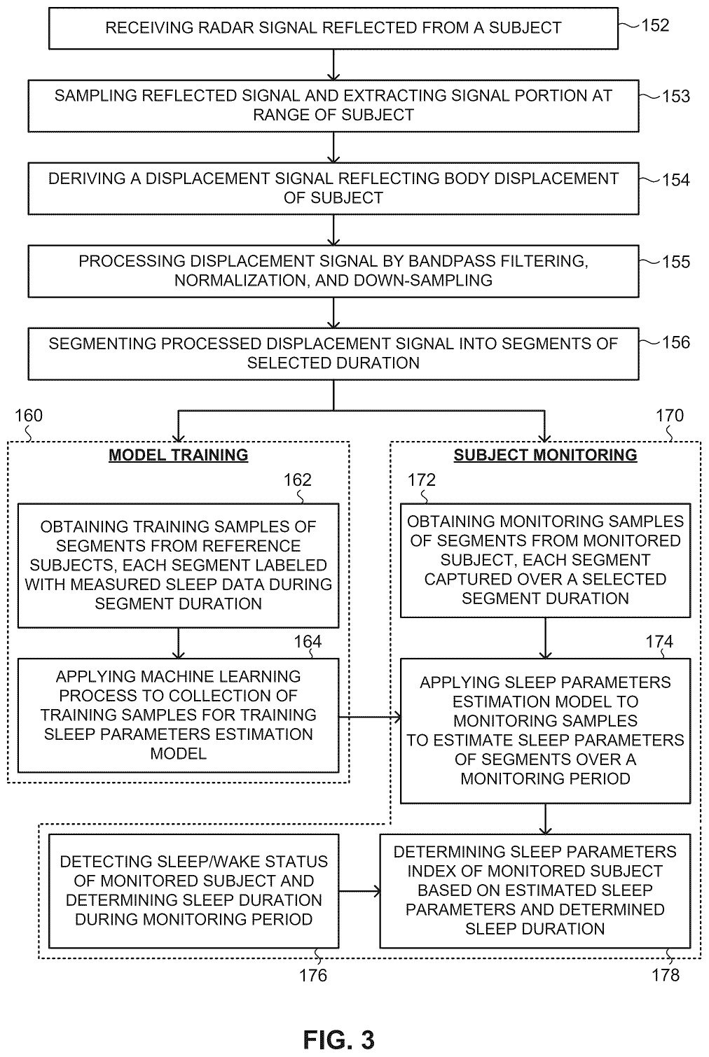

FIG. 3 illustrates a block diagram of a sleep parameters monitoring method according to aspects of the present disclosure. A comprehensive sleep parameters monitoring method may be implemented through a systematic approach that processes radar signals to generate sleep parameter assessments. The method may begin with a step 152 of receiving a radar signal reflected from a subject. Referring to FIGS. 1 and 2, radar device 112 transmits a transmitted radar signal 122, such as a FMCW THz radar signal, and receives a corresponding reflected radar signal 124 from subject 120, where reflected radar signal 124 may contain physiological information related to cardiac and pulmonary activities that may be extracted through subsequent processing operations. A next step 153 involves sampling the reflected radar signal and extracting a signal portion at the range of the subject.

Processor 114 receives and filters reflected radar signal 124 based on signal phase differences that correlate with distance, to isolate signal components at the range at which subject 120 is located. Processor 114 may receive the reflected radar signal 124, obtained at a selected sampling rate and recorded with two channels consisting of an in-phase (I) component and a quadrature (Q) component, and measures a phase difference between the transmitted signal 122 and received signal 124 to determine the distance traversed by the radar signal, so as to extract the signal portion corresponding to reflections from subject 120 and removing noise and irrelevant signal components at other ranges. For example, if transmitted and reflected radar signals 122, 124 are FMCW radar signals, then processor 114 may apply a fast Fourier transform (FFT) to extract the signal portion corresponding to the subject range. For example, if operating in FMCW mode the reflected radar signal is sampled at a selected rate, e.g., 500 Hz, such that 500 times per second a vector of multiple samples (e.g., 128 samples) is collected (e.g., providing 64 kHz samples per second). This received signal (e.g., of 128 samples) undergoes a Fourier transform, such that each transformed sample is respective of a range. Of these transformed samples, an individual sample corresponding to the subject range is extracted, resulting in a collection of values in accordance with the sampling rate (e.g., 500 extracted subject range samples per second).

The method may continue with a step 154 where a displacement signal reflecting body displacement of the subject is derived from the processed radar signal. The displacement signal may characterize repetitive body micromovements resulting from blood flow, cardiac ejection forces, and chest motion from respiratory activities. Processor 114 may apply non-linear filtering or mapping operations to the extracted radar signal portion with I/Q components to obtain the displacement signal that represents physiological micro-displacements. An optional next step 155 involves processing the displacement signal to prepare the signal for segmentation and analysis. The processing may include bandpass filtering, normalization, and/or down-sampling operations. The bandpass filtering may remove very low and very high frequency components, for example beyond a range of approximately 0.05 Hz to 3.33 Hz, while preserving frequencies pertaining to vital signs such as respiration, heart rate, and various harmonics or derivatives of physiological phenomena. The down-sampling may convert the signal to a reduced sampling rate, such as from 500 Hz to 10 Hz. The processing operations of bandpass filtering, normalization, and down-sampling may be executed in different sequential orders.

As further shown in FIG. 3, a next step 156 includes segmenting the processed displacement signal into segments of a selected time duration, to produce vectors of predetermined length for machine learning analysis. The segmentation process may divide the processed signal into time-based segments that may serve as input data for subsequent model training or subject monitoring operations. For example, if the down-sampled frequency of the displacement signal is 10 Hz, then a segmentation of 15 minutes provides a vector data set of length: 15×60 seconds×10 Hz=9000. The segmentation duration may be an adjustable parameter. In some cases, system 110 may process consecutive segments with overlapping durations to enhance temporal resolution and analysis accuracy. The overlapping segment processing may involve creating segments where consecutive time periods may share common portions, such as 10-second segments that may overlap by 9 seconds, allowing for more granular analysis of physiological patterns while maintaining computational efficiency. The overlapping approach may provide enhanced detection capabilities by ensuring that physiological events occurring near segment boundaries may be captured and analyzed across multiple overlapping time windows.

The method may branch into parallel processing architectures that may operate simultaneously to achieve comprehensive sleep parameter monitoring capabilities. A model training phase 160 may be implemented to develop machine learning models using reference data from multiple subjects, while a monitoring phase 170 may apply the trained models to assess sleep parameters of monitored subjects. The parallel processing architecture may enable the system 110 to continuously update and refine machine learning models while simultaneously providing real-time sleep parameter assessments. In some cases, the model training phase 160 may operate on historical data collected from reference subjects, while the monitoring phase 170 may process real-time data from current monitoring sessions, allowing for dynamic model improvement and immediate sleep parameter evaluation.

The model training phase 160 includes a step 162 of obtaining training samples of segments from reference subjects, with each segment labeled with measured sleep data during the segment duration. The training samples may be derived from radar signal processing of multiple reference subjects who may be simultaneously monitored using PSG device 116 to provide ground truth physiological measurements. In a next step 164, machine learning processes are applied to the collection of training samples to train a sleep parameters estimation model for predicting sleep parameters from radar-derived displacement signals. The machine learning training may utilize various algorithms, such as artificial neural networks, regression analysis, classification models, or decision tree learning approaches, to establish mapping functions between radar signal characteristics and sleep parameter indicators.

The monitoring phase 170 implements real-time sleep parameter assessment through a series of coordinated processing steps that may apply trained machine learning models to current monitoring data. In a step 172, monitoring samples of segments are obtained from a monitored subject, where each segment may be captured over the selected segment duration that corresponds to the training data segment length. The monitoring samples may be processed continuously as radar data may be acquired from a monitored subject 120 during sleep monitoring sessions. In some cases, system 110 may detect subject occupancy using object detection sensors, proximity sensors, or motion sensors to determine presence or absence of the monitored subject 120, enhancing monitoring accuracy by confirming subject presence and distinguishing between occupied and unoccupied monitoring periods. The occupancy detection may provide additional validation for radar signal interpretation and may prevent false readings when subject 120 may be absent from the monitoring area.

A next step 174 involves applying the sleep parameters estimation model to the monitoring samples to estimate sleep parameters of segments over a monitored time period. The trained machine learning model may analyze the displacement signal characteristics within each segment to predict sleep-related parameters such as apnea events, sleep stages, or other physiological indicators. In a next step 176, the sleep status of the monitored subject is detected and an overall sleep duration during the monitoring period is determined. Processor 114 may distinguish between sleep and wake states to calculate the total time spent in actual sleep. The sleep status detection may utilize supplemental information from motion sensors, proximity sensors, or other detection systems to enhance accuracy of sleep duration calculations. In a further step 178, a sleep parameters assessment of the monitored subject is determined based on the estimated sleep parameters and the determined sleep duration, providing quantitative metrics such as apnea-hypopnea index values or sleep stage distribution percentages that may be used for clinical assessment or sleep quality evaluation.

The sleep parameters monitoring method of FIG. 3 may operate continuously and/or iteratively, such that at least some of the steps are performed repeatedly, to provide for continuous sleep monitoring of a subject 120 over a monitoring duration.

### Data Acquisition and Machine Learning Training

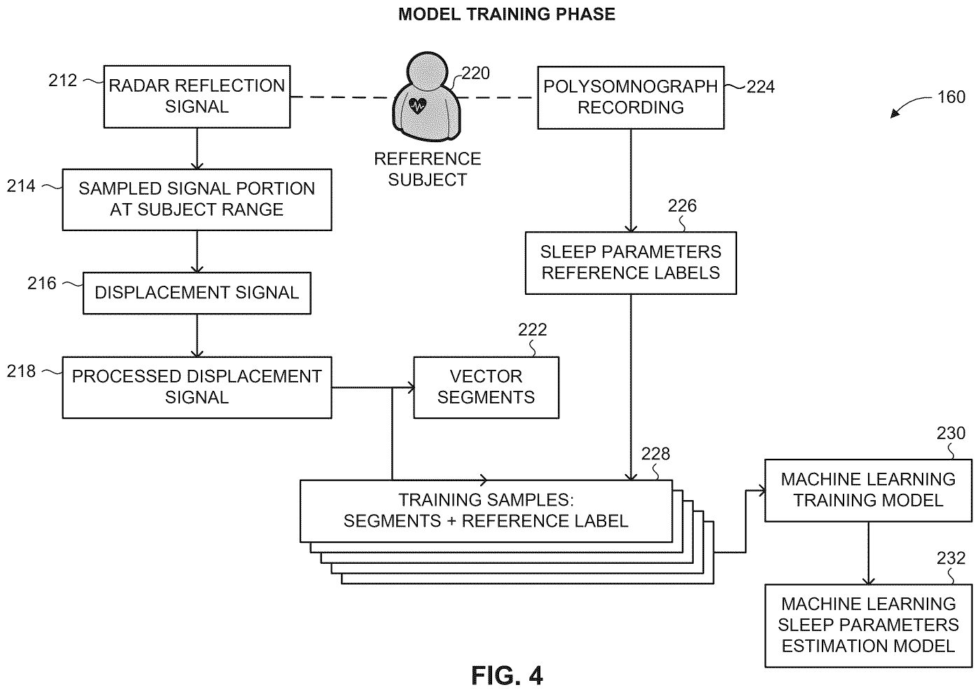

Referring to FIG. 4, the model training phase 160 may implement a comprehensive data acquisition and machine learning training workflow that transforms radar-based physiological measurements into a predictive model for sleep parameter estimation. During the model training phase 160, system 110 may acquire training data from multiple reference subjects to establish robust machine learning models capable of accurately predicting sleep parameters from radar signal characteristics. The training process begins when a radar reflection signal 212 is obtained from a reference subject 220 using radar device 112. The radar reflection signal 212 may contain embedded physiological information related to cardiac and pulmonary activities that may be extracted through systematic signal processing operations. Radar reflection signal 212 may be obtained using FMCW radar operating in THz or MMW frequency bands to ensure adequate penetration through clothing and bedding materials while maintaining signal integrity for physiological detection.

A sampled signal portion 214 is derived from radar reflection signal 212 through sampling and filtering operations that isolate signal components at the range where reference subject 220 may be positioned. The sampling process may operate at a selected sampling rate, such as approximately 500 Hz, to capture high-resolution temporal variations in the reflected radar signals, where the sampled signal portion 214 may consist of in-phase (I) and quadrature (Q) components that preserve both amplitude and phase information from the radar reflections. Processor 114 may apply fast Fourier transform operations to radar reflection signal 212 to extract the sampled signal portion 214 corresponding to the specific range bin where reference subject 220 is located, effectively filtering out reflections from other distances and environmental objects.

A displacement signal 216 is derived from sampled signal portion 214 through non-linear filtering and mapping operations that convert the complex-valued radar data into physiological displacement measurements. Displacement signal 216 may represent repetitive body micromovements resulting from ballistic forces associated with cardiac ejection, blood flow patterns, and respiratory chest motion. Processor 114 may apply mathematical transformations to the in-phase (I) and quadrature (Q) components of sampled signal portion 214 to calculate displacement values that correspond to physiological micro-movements of reference subject 220. Displacement signal 216 may be computed through angle calculations of complex numbers formed by the in-phase (I) and quadrature (Q) signal components, where the resulting displacement values may reflect temporal variations in body surface positions caused by internal physiological processes.

Displacement signal 216 may be processed through signal conditioning operations to produce a processed displacement signal 218 that may be optimized for machine learning analysis. The signal processing operations may include bandpass filtering that removes frequency components outside a selected range, such as outside a range between 0.05 Hz and 3.33 Hz, where the filtering may preserve frequencies pertaining to vital signs such as respiration, heart rate, and various harmonics or derivatives of physiological phenomena while eliminating low-frequency drift and high-frequency noise. The processed displacement signal 218 may undergo normalization operations that standardize signal amplitudes across different reference subjects and monitoring sessions, ensuring consistent input characteristics for machine learning model training. Down-sampling operations may reduce the sampling rate, such as from 500 Hz to 10 Hz, to decrease computational requirements while maintaining adequate temporal resolution for physiological pattern recognition, where the down-sampling process may apply anti-aliasing filters to prevent frequency folding artifacts.

The processed displacement signal 218 may be segmented into time-based vectors to create a reference dataset 222 that serves as input data for machine learning model training. The segmentation process may divide the processed displacement signal 218 into segments of a selected temporal duration, such as 15-minute segments, where each segment may be converted into a vector of selected size, such as a vector length calculated as 15×60 secondsx10 Hz=9000 data points. The mathematical relationship for vector length calculation may ensure that each training sample contains sufficient temporal information to capture physiological patterns while maintaining computational tractability for machine learning algorithms. In some cases, the reference dataset 222 may include vectors from multiple reference subjects monitored over extended periods, where each vector may represent a discrete time segment that may be independently analyzed for sleep parameter characteristics. The segmentation approach may enable the machine learning models to learn associations between radar signal patterns and sleep parameters at consistent temporal resolutions across different subjects and monitoring sessions.

A polysomnograph (PSG) recording 224 may be obtained from reference subject 220 using PSG device 116 to provide ground truth physiological measurements that correspond to the radar-derived displacement signals. The polysomnograph recording 224 may include electrocardiography measurements for cardiac activity, electroencephalography measurements for brain activity, electrooculography measurements for eye movements, and/or electromyography measurements for muscle activity, which characterizes the sleep state and sleep events of the reference subject 220. Sleep parameters reference labels 226 may be assigned to each vector in the reference dataset 222 based on information extracted from the polysomnograph recording 224, where the reference labels may reflect measured sleep data during the segment duration corresponding to each vector. The sleep parameters reference labels 226 may include quantitative measurements such as the number of apnea or hypopnea events detected during each segment, predominant sleep stage classifications for each segment, or binary indicators for the presence or absence of specific physiological phenomena.

In some cases, the sleep parameters reference labels 226 may include apnea event counts that quantify the number of respiratory interruptions detected during each time segment, and/or sleep stage classifications that identify the predominant sleep phase experienced by the reference subject 220 during the corresponding time period. For example, a vector segment of one-minute duration may include 60 entries for each second, where each entry is assigned an apnea state indication (e.g., positive or negative) for whether an apnea event occurred during that specific second. An overall apnea count may then be assigned to the entire vector segment based on the number of positive apnea event indications throughout the segment. For example, if an apnea event occurred during 10 of the 60 seconds of a one-minute segment then the segment may be assigned an apnea event count of “10”. The apnea event count may represent at least part of a reference label of the training sample for subsequent model training. In another example, the reference sleep data includes sleep stages information, such as detected sleep stages during the segment duration. Each vector segment may be assigned an indication of the predominant sleep stage experienced by the reference subject (i.e., sleep stage in which most time was spent) during the segment duration, as determined based on PSG measurements. Specifically, one or more vector indices of the segment may be assigned a sleep stage indication reflecting the sleep stage detected during the respective time duration. For example, a vector segment of one-minute duration may include 60 entries for each second, where each entry is assigned a sleep stage indication (e.g., REM, N1, N2, N3, or awake) based on a detected sleep stage of the reference subject during that specific second. An overall sleep stage indication may then be assigned to the entire vector segment based on the predominant sleep stage throughout the segment. For example, a one-minute segment may include 5 seconds of a detected “awake” state, 15 second of a detected “REM sleep” state, and 40 seconds of a detected “N1 sleep” state, in which case the segment may be assigned a sleep stage indication of “N1”. The sleep stage indication may represent at least part of a reference label of the training sample for subsequent model training. In one example, a reference label of a training sample includes both sleep apnea information (e.g., apnea event count for segment) and sleep stage information (e.g., sleep stage indication for segment).

With continued reference to FIG. 4, training samples 228 are formed by combining vectors from reference dataset 222 with their corresponding sleep parameters reference labels 226 to create labeled datasets for supervised machine learning. Each training sample within the training samples 228 may consist of a vector (e.g., 9000-element vector) representing radar-derived physiological measurements paired with quantitative or categorical labels that describe the sleep parameters during the corresponding time segment. The training samples 228 may originate from various physiological measurement sources beyond PSG device 116 to enhance training data diversity and model robustness. In some cases, training samples 228 may incorporate data from accelerometers or motion sensors worn on body parts such as the chest, back, or abdomen that provide complementary movement measurements. Pressure sensors situated in or under lying surfaces such as beds or mattresses may contribute additional training data that reflects body position changes and respiratory movements, while other contactless radar devices may provide alternative radar-based measurements that augment the primary radar sensing data.

A machine learning training model 230 may process the training samples 228 through various algorithmic approaches to identify patterns and relationships between radar signal characteristics and sleep parameters. For example, the machine learning training model 230 may be implemented using artificial neural networks including convolutional neural networks that analyze spatial and temporal patterns in the displacement signal data, or recurrent neural networks that capture sequential dependencies in physiological time series. Deep learning algorithms may be applied to automatically extract hierarchical features from the radar signal data without requiring manual feature engineering, while support-vector machine models may establish decision boundaries for classification tasks such as sleep stage identification. In some cases, decision tree learning approaches such as random forest classifiers may be utilized to create ensemble models that combine multiple decision trees for enhanced prediction accuracy, or combinations of different algorithmic approaches may be integrated to leverage the strengths of various machine learning techniques. The machine learning training model 230 may implement a dual neural network architecture that provides complementary approaches to sleep parameter estimation through parallel processing pathways. A regression neural network component may be configured to estimate numerical values of respiratory events by learning mapping functions between radar signal patterns and quantitative measures such as apnea event counts per time segment. The regression neural network may output continuous numerical predictions that correspond to the frequency or severity of respiratory disturbances detected in the radar signal data. A segmentation neural network component may operate in parallel to determine for every second of each time segment whether the corresponding radar signal characteristics belong to a respiratory event or normal breathing pattern. In some cases, the segmentation neural network may provide binary classifications for each second within the overall (e.g., 15-minute) temporal segments, generating detailed temporal maps of respiratory event occurrences that may be aggregated to produce overall segment-level assessments.

The machine learning training model 230 may establish classification profiles by assigning reference subjects into different groups based on demographic and physiological characteristics that may influence sleep parameter patterns. The classification profiles may categorize reference subjects according to age ranges, gender classifications, geographic locations, and physical attributes such as body mass index, height, or weight that may affect radar signal propagation and physiological signal characteristics. Each classification profile may be associated with relative weighting metrics that correspond to confidence levels pertaining to the respective category features, where the weighting metrics may quantify the reliability of sleep parameter predictions for subjects belonging to specific demographic or physiological groups. Processor 114 may apply different model parameters or feature extraction approaches for different classification profiles, enabling customized sleep parameter estimation that accounts for population-specific variations in physiological patterns and radar signal characteristics.

The machine learning training model 230 may generate and dynamically update a machine learning sleep parameters model 232 that encapsulates the learned relationships between radar signal patterns and sleep parameters across the training dataset. The machine learning sleep parameters model 232 may incorporate the trained neural network weights, decision boundaries, feature extraction parameters, and classification profile information that enable accurate sleep parameter prediction for new radar signal data. The model generation process may involve iterative optimization procedures that minimize prediction errors across the training samples 228 while preventing overfitting through regularization techniques or cross-validation approaches. The machine learning sleep parameters model 232 may be continuously updated as additional training data becomes available from new reference subjects or extended monitoring sessions, allowing the model to adapt to evolving datasets and improve prediction accuracy over time through incremental learning processes.

### Subject Monitoring and Sleep Parameters Analysis

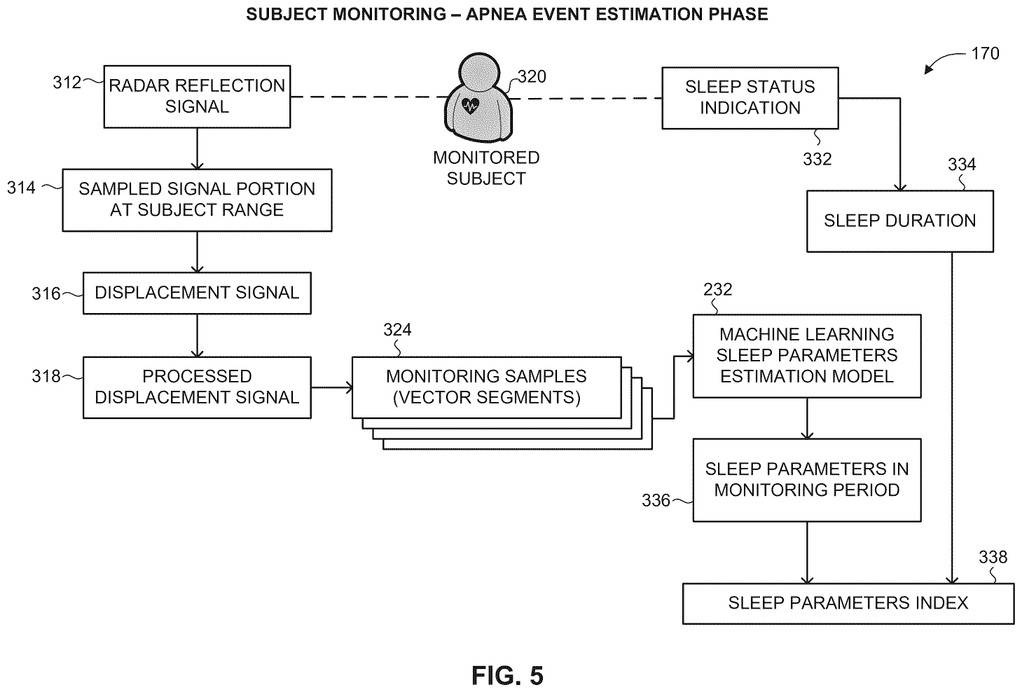

Referring to FIG. 5, the monitoring phase 170 may implement comprehensive subject monitoring and sleep parameters analysis through systematic processing of radar-based physiological measurements obtained from monitored subjects during sleep sessions. The subject monitoring phase 170 begins when a radar reflection signal 312 is obtained from a monitored subject 320 using radar device 112 during sleep monitoring periods. Radar reflection signal 312 may contain physiological information related to cardiac and pulmonary activities that may be extracted through signal processing operations similar to those applied during the model training phase 160. Radar reflection signal 312 may be obtained using FMCW radar operating in THz or MMW frequency bands, where the high-frequency electromagnetic waves may penetrate through clothing and bedding materials while maintaining signal integrity for physiological detection. Radar reflection signal 312 may be continuously acquired throughout the monitoring session to provide comprehensive temporal coverage of sleep-related physiological patterns. Radar device 112 may provide detection of subtle body surface displacements caused by internal physiological processes without requiring direct contact with monitored subject 320, allowing for continuous monitoring throughout sleep periods without disturbing natural sleep patterns.

A sampled signal portion 314 is derived from radar reflection signal 312 through sampling and range extraction operations that isolate signal components corresponding to the position of the monitored subject 320. The sampling process may operate at a selected sampling rate, such as approximately 500 Hz, to capture temporal variations in the reflected radar signals with adequate resolution for physiological pattern detection and to maintain consistency with the training data characteristics. The sampled signal portion 314 may consist of in-phase (I) and quadrature (Q) components that preserve both amplitude and phase information from the radar reflections, where the complex-valued signal data may enable accurate distance measurements and physiological displacement calculations. Fast Fourier transform operations may be applied to the radar reflection signal 312 to extract the sampled signal portion 314 corresponding to the specific range bin where the monitored subject 320 may be located, effectively filtering out other environmental reflections and noise components. The range extraction process may dynamically adjust to account for subject movement during sleep, ensuring continuous tracking of monitored subject 320 throughout the monitoring session.

A displacement signal 316 is derived from sampled signal portion 314 through transformations that convert the complex-valued radar data into physiological displacement measurements representing body micro-movements. Displacement signal 316 may characterize repetitive physiological motions resulting from cardiac ejection forces, blood flow patterns, and respiratory chest movements. Processor 114 may apply non-linear filtering and mapping operations to the in-phase (I) and quadrature (Q) components of sampled signal portion 314 to calculate displacement values that correspond to temporal variations in body surface positions caused by internal physiological processes. Displacement signal 316 may undergo the same processing operations that were applied during the model training phase 160 to ensure consistency between training and monitoring data characteristics, where angle calculations of complex numbers formed by the in-phase (I) and quadrature (Q) signal components may produce displacement values that reflect physiological micro-movements with temporal resolution suitable for sleep parameter analysis.

Displacement signal 316 may be processed through signal conditioning operations to produce a processed displacement signal 318 that may be optimized for analysis by the machine learning sleep parameters model 232. The signal processing operations may include bandpass filtering that removes frequency components outside a selected range, such as outside a range between 0.05 Hz and 3.33 Hz, where the filtering may preserve frequencies pertaining to vital signs while eliminating low-frequency drift and high-frequency noise. The processed displacement signal 318 may undergo normalization operations that standardize signal amplitudes to match the characteristics of the training data used to develop the machine learning sleep parameters model 232, ensuring consistent input parameters for accurate sleep parameter prediction. Down-sampling operations may reduce the sampling rate, such as from 500 Hz to 10 Hz, to match the temporal resolution used during model training, where anti-aliasing filters may be applied to prevent frequency folding artifacts that could degrade signal quality. The processed displacement signal 318 may be continuously generated throughout the monitoring session, providing real-time physiological data that may be segmented and analyzed for sleep parameter estimation.

The processed displacement signal 318 may be segmented into time-based vectors that form monitoring samples 324 for input to the machine learning sleep parameters model 232 during sleep parameter assessment. The segmentation process may divide the processed displacement signal 318 into segments of a temporal duration that corresponds to the segment length used during model training, such as 15-minute segments, where each segment may be converted into a vector of selected size. For example, segments of 15-minute duration sampled at 10 Hz may produce vectors of 9000 data points calculated as 15×60 secondsx10 Hz=9000, ensuring compatibility with the machine learning sleep parameters model 232 that was trained using vectors of identical dimensions. The monitoring samples 324 may be processed continuously as radar data may be acquired from the monitored subject 320, enabling real-time sleep parameter estimation throughout extended monitoring periods such as overnight sleep sessions. In some cases, consecutive segments may be processed with overlapping durations to enhance temporal resolution and ensure that physiological events occurring near segment boundaries may be captured and analyzed across multiple time windows.

The machine learning sleep parameters model 232 may analyze the monitoring samples 324 to generate sleep parameter estimates that quantify various aspects of sleep quality and respiratory function during the monitoring period. The sleep parameter estimation may utilize machine learning processes to predict sleep parameters such as apnea event counts or sleep stage classifications based on the learned relationships between radar signal patterns and physiological indicators established during the model training phase 160. For example, machine learning sleep parameters model 232 may estimate numerical values representing the frequency of sleep apnea events within each time segment, and/or provide detailed temporal classifications that identify specific times within each segment where respiratory disturbances may occur. The sleep parameter estimates may be generated for each monitoring sample 324 and aggregated across the entire monitoring period to provide comprehensive assessments of sleep quality and respiratory function. A monitoring period may extend over entire nighttime sleep sessions, typically ranging from several hours to complete overnight periods depending on the monitoring objectives and subject sleep patterns.

A sleep status indication 332 may be obtained to distinguish between sleep and wake states of the monitored subject 320 throughout the monitoring period, enabling accurate calculation of total sleep duration for sleep parameter indices. The sleep status indication 332 may be obtained using PSG device 116, such as derived from PSG recording 224, or obtained from an alternate source. In some cases, the sleep status indication 332 may be generated through analysis of the radar signal characteristics themselves, where processor 114 may identify patterns in the displacement signal that correspond to sleep versus wake states based on movement patterns, breathing regularity, or other physiological indicators. Sleep status indication 332 may be derived based on a signal reflecting the presence or absence of monitored subject 320, such as from object detection sensors, proximity sensors, or motion sensors that provide validation of subject presence and activity levels during the monitoring period. For example, if subject motion is detected and exceeds a certain threshold then it may indicate that the subject is in a wakened state. Processor 114 may analyze the sleep status indication 332 to identify periods when the monitored subject 320 may be absent from the monitoring area, where absence detection may prevent false sleep parameter calculations and provide status indicators such as “absent” or “empty” during unoccupied periods. Sleep status indication 332 may provide temporal information indicating specific time periods when the monitored subject 320 may be in active sleep states versus periods of wakefulness or absence from the monitoring area.

A sleep duration 334 may be calculated based on sleep status indication 332 to quantify the total time spent in actual sleep during the monitoring period, excluding periods of wakefulness or subject absence. Processor 114 may analyze the sleep status indication 332 to identify continuous periods of sleep and calculate cumulative sleep time measurements that account for sleep interruptions, wake periods, or temporary absence from the monitoring area. The sleep duration 334 calculation may involve temporal integration of sleep state periods while excluding time segments where the monitored subject 320 may be determined to be awake or absent based on the sleep status indication 332. In some cases, sleep duration 334 may be expressed in hours, minutes, and seconds to provide precise temporal measurements that enable accurate calculation of sleep parameter indices such as apnea-hypopnea index values.

Sleep parameters in a monitoring period 336 may be determined by aggregating the sleep parameter estimates generated by machine learning sleep parameters model 232 across all monitoring samples 324 collected during the monitoring session. The sleep parameters in the monitoring period 336 may include quantitative measurements such as total counts of apnea events, hypopnea events, or other respiratory disturbances detected throughout the monitoring session, as well as qualitative assessments such as sleep stage distributions or sleep quality indicators. Processor 114 may combine the individual segment-level sleep parameter estimates to generate comprehensive assessments that characterize the overall sleep experience of the monitored subject 320 during the monitoring period. The aggregation process may account for temporal variations in sleep parameters throughout the night, providing detailed information about sleep architecture and respiratory function patterns that may vary across different sleep stages or time periods.

A sleep parameters index 338 may be calculated based on the sleep parameters in the monitoring period 336 and the sleep duration 334 to provide standardized metrics that enable clinical assessment and comparison across different monitoring sessions. The sleep parameters index 338 may include apnea-hypopnea index (AHI) calculations that quantify the frequency of apnea events per hour of sleep, where the index may be computed using the mathematical formula: AHI=(total apnea events+total hypopnea events)/(overall sleep duration in hours). The apnea-hypopnea index values may be classified according to established severity level categories. In an exemplary categorization of sleep apnea severity for an adult, AHI<5 indicates normal respiratory function, 5≤AHI<15 indicates mild sleep apnea, 15≤AHI<30 indicates moderate sleep apnea, and AHI≥30 indicates severe sleep apnea conditions. The sleep parameters index 338 may also include a sleep stage index, such as sleep stage distribution metrics that quantify the total time and/or percentage of sleep time spent in different sleep stages during the monitoring session. For example, the sleep stage index may characterize the time spent in: REM sleep, NREM stage 1, NREM stage 2, NREM stage 3, and wake states. If a given segment contains an amount of wake (non-sleep) time that exceeds a certain threshold (e.g., a high percentage of seconds in which the subject is in a wakened state), then that segment may be removed from the derived sleep parameters index 338. In some cases, the sleep parameters index 338 may incorporate additional metrics such as sleep efficiency percentages, sleep onset latency measurements, and wake after sleep onset calculations that provide comprehensive assessment of sleep quality and sleep architecture characteristics.

System 110 may provide behavioral recommendations for the monitored subject 320 to alleviate sleep disorders and enhance sleep quality based on statistical analysis across multiple monitoring sessions conducted over extended periods. The behavioral recommendation may analyze longitudinal data collected from repeated monitoring sessions to identify patterns, trends, or correlations between sleep parameters and external factors such as bedtime routines, environmental conditions, or lifestyle factors that may influence sleep quality. Processor 114 may calculate statistical measures such as average apnea-hypopnea index values, sleep stage distribution variations, peak respiratory event frequencies, and standard deviations across multiple monitoring sessions to establish baseline sleep characteristics and identify areas for improvement. The statistical analysis may encompass data collected over periods of several days, weeks, or months to provide comprehensive assessments of sleep patterns and identify temporal variations that may be associated with lifestyle changes, treatment interventions, or environmental factors.

The behavioral recommendations may be generated through analysis of correlations between sleep parameter variations and external factors such as sleep timing, environmental conditions, physical activity levels, or dietary patterns that may be recorded through supplemental data collection systems or user input interfaces. Database 118 may store historical sleep parameter data, environmental measurements, and lifestyle information that enable processor 114 to identify relationships between modifiable factors and sleep quality improvements. The recommendation system may utilize machine learning algorithms to analyze the longitudinal data and generate personalized suggestions for sleep hygiene improvements, lifestyle modifications, or environmental adjustments that may reduce sleep apnea severity or enhance overall sleep quality. In some cases, the behavioral recommendations may include specific guidance regarding sleep positioning, bedroom temperature control, exercise timing, or dietary modifications that may be tailored to the individual sleep patterns and physiological characteristics of the monitored subject 320 based on the accumulated monitoring data and statistical analysis results. The behavioral recommendations may be generated through comparison of individual sleep parameter patterns with population-based norms or clinical guidelines that define healthy sleep characteristics for different demographic groups. System 110 may identify specific sleep parameter abnormalities or trends that may indicate underlying sleep disorders, where targeted recommendations may address factors such as sleep hygiene practices, bedroom environment modifications, or lifestyle changes that may improve sleep quality and reduce sleep disorder symptoms. The behavioral recommendations may be continuously updated as additional monitoring data becomes available, enabling personalized sleep improvement strategies that adapt to changing sleep patterns and treatment responses over time.

The disclosed embodiments provide reliable and accurate assessment of sleep parameters, including sleep apnea severity through AHI metrics and time spent in different sleep stages, as determined through advanced machine learning processes. Sleep parameters and calculated statistics obtained over multiple monitoring sessions enable comprehensive evaluation and form the basis for targeted recommendations to alleviate sleep disorders and enhance sleep quality. The disclosed system and method utilize contactless operation, eliminating the need for physical sensors or devices to be coupled to the subject's body prior to monitoring sessions, thereby saving time and minimizing patient discomfort. The system operates effectively even when the subject is not directly visible to the radar device, functioning reliably under poor visibility or light saturation conditions. The radar signals can penetrate obstructions such as clothing or blankets and operate from various angles relative to the subject, while source separation techniques enable concurrent monitoring and identification of multiple subjects. The system architecture requires minimal costly equipment, incorporates relatively few components, and provides straightforward operation and maintenance. The disclosed method and system may demonstrate exceptional versatility, operating effectively across diverse locations, sleeping surfaces of various types and sizes, and subjects with different physical characteristics, all without requiring time-consuming calibration processes for each monitoring session. The machine learning analysis may continuously improve through iterative refinement, enhancing the accuracy of sleep parameter estimation for new subjects based on accumulated data and experience.

Aspects of the present disclosure can be utilized in various applications for monitoring patients to evaluate and improve sleep quality, such as in a home setting or in a healthcare facility.

While certain embodiments of the present disclosure have been described, the preceding description is intended to be exemplary only. It will be understood that various modifications may be made without departing from the spirit and scope of the disclosure. Accordingly, other implementations are within the scope of the following claims.

1. A method for monitoring sleep parameters, comprising:
during a model training phase, for each of a plurality of reference subjects:
receiving a millimeter-wave reflection radar signal reflected from a respective one of the reference subjects;
sampling the reflection radar signal and extracting a signal portion at a range of the respective one of the reference subjects, the signal portion consisting of an in-phase component and a quadrature component;
deriving a displacement signal reflecting body micromovements associated with cardiac and pulmonary activity from the extracted signal portion;
segmenting the displacement signal into reference subject segments having a selected segment duration;
forming a training dataset comprising training samples, each training sample comprising a respective reference subject segment labeled with measured sleep data during the segment duration; and
applying machine learning processes to the training dataset to generate a sleep parameters estimation model, and
during a subject monitoring phase:
receiving a millimeter-wave reflection radar signal reflected from a monitored subject;
sampling the reflection radar signal and extracting a signal portion at a range of the monitored subject, the signal portion consisting of an in-phase component and a quadrature component;
deriving a displacement signal reflecting body micromovements associated with cardiac and pulmonary activity from the extracted signal portion;
segmenting the displacement signal into monitored subject segments having the selected segment duration;
applying the sleep parameters estimation model to the monitored subject segments to estimate sleep parameters of the monitored subject over a monitoring period;
determining an overall sleep duration of the monitored subject during the monitoring period; and
determining a sleep parameters index of the monitored subject based on the estimated sleep parameters and the determined sleep duration.

2. The method of claim 1, wherein the sleep parameters comprise at least one of: sleep apnea events; and sleep stage classifications.

3. The method of claim 1, wherein the sleep parameters index comprises at least one of:
an apnea-hypopnea index calculated as a total number of detected apnea and hypopnea events divided by the overall sleep duration; and
a sleep stage distribution index.

4. The method of claim 1, further comprising processing the displacement signal prior to segmenting, using at least one of: bandpass filtering; normalization; and down-sampling.

5. The method of claim 4, comprising applying bandpass filtering to the displacement signal to preserve frequencies between 0.05 Hz and 3.33 Hz.

6. The method of claim 4, wherein the reflection radar signal is sampled at a sampling rate of 500 Hz and the displacement signal is down-sampled to 10 Hz prior to the segmenting.

7. The method of claim 1, wherein the displacement signals are derived from the extracted signal portions through non-linear filtering and mapping operations applied to the in-phase and quadrature components.

8. The method of claim 1, wherein determining the overall sleep duration comprises detecting a sleep status of the monitored subject to distinguish between sleep and wake states during the monitoring period.

9. The method of claim 1, wherein the millimeter-wave reflection radar signal comprises a frequency-modulated continuous wave (FMCW) radar signal.

10. The method of claim 9, wherein extracting a signal portion comprises applying a fast Fourier transform (FFT) to the FMCW radar signal.

11. The method of claim 1, wherein the millimeter-wave reflection radar signal is at a frequency above 100 GHz.

12. The method of claim 1, further comprising establishing classification profiles by categorizing reference subjects based on demographic and physiological characteristics, and applying different model parameters for different classification profiles during sleep parameter estimation.

13. A system for monitoring sleep parameters, comprising:
a radar device configured to receive a millimeter-wave reflection radar signal reflected from each of a plurality of reference subjects during a model training phase, and to receive a millimeter-wave reflection radar signal reflected from a monitored subject during a subject monitoring phase; and
a processor configured to, during the model training phase, for each of the plurality of reference subjects:
sample the reflection radar signal reflected from the respective one of the reference subjects and extract a signal portion at a range of the respective one of the reference subjects, the signal portion consisting of an in-phase component and a quadrature component;
derive a displacement signal reflecting body micromovements associated with cardiac and pulmonary activity from the extracted signal portion;
segment the displacement signal into reference subject segments having a selected segment duration;
form a training dataset comprising training samples, each training sample comprising a respective reference subject segment labeled with measured sleep data during the segment duration; and
apply machine learning processes to the training dataset to generate a sleep parameters estimation model, and
during the subject monitoring phase:
sample the reflection radar signal reflected from the monitored subject and extract a signal portion at a range of the monitored subject, the signal portion consisting of an in-phase component and a quadrature component;
derive a displacement signal reflecting body micromovements associated with cardiac and pulmonary activity from the extracted signal portion;
segment the displacement signal into monitored subject segments having the selected segment duration;
apply the sleep parameters estimation model to the monitored subject segments to estimate sleep parameters of the monitored subject over a monitoring period;
determine an overall sleep duration of the monitored subject during the monitoring period; and
determine a sleep parameters index of the monitored subject based on the estimated sleep parameters and the determined sleep duration.

14. The system of claim 13, wherein the sleep parameters comprise at least one of: sleep apnea events; and sleep stage classifications.

15. The system of claim 13, wherein the sleep parameters index comprises at least one of:
an apnea-hypopnea index calculated as a total number of detected apnea and hypopnea events divided by the overall sleep duration; and
a sleep stage distribution index.

16. The system of claim 13, wherein the processor is further configured to process the displacement signal prior to segmenting, using at least one of: bandpass filtering; normalization; and down-sampling.

17. The system of claim 13, wherein the processor is configured to determine the overall sleep duration by detecting a sleep status of the monitored subject to distinguish between sleep and wake states during the monitoring period.

18. The system of claim 13, wherein the processor is configured to determine the overall sleep duration by detecting a sleep status of the monitored subject to distinguish between sleep and wake states during the monitoring period.

19. The system of claim 13, wherein the radar device is configured to receive the millimeter-wave reflection radar signal as a frequency-modulated continuous wave (FMCW) radar signal.

20. The system of claim 13, wherein the processor is configured to establish classification profiles by categorizing reference subjects based on demographic and physiological characteristics, and apply different model parameters for different classification profiles during sleep parameter estimation.
